(19) United States
(12) Patent Application Publication
(10) Pub. No.: US 2005/0239029 A1
(43) Pub. Date: Oct. 27, 2005

(54) Method of enunciating a prerecorded message related to toilet training in response to a contact

(76) Inventors: Erin K. Yzermans, Menasha, WI (US); Christa M. Debbout, Appleton, WI (US); Carmen E. Van Dam, Appleton, WI (US); Katherine Krenger, Chicago, IL (US); Melissa D. Maher, Chicago, IL (US); Denise M. Young, Chicago, IL (US)

Correspondence Address: KIMBERLY-CLARK WORLDWIDE, INC., 401 NORTH LAKE STREET, NEENAH, WI, 54956, US

(21) Appl. No.: 10/831,476

(22) Filed: Apr. 23, 2004

(51) Int. Cl. G09B 19/00; A61F 13/15; A61F 13/20

(52) U.S. Cl. 434/258

(57) ABSTRACT

A method of enunciating a prerecorded message related to toilet training in response to a contact is disclosed. In particular embodiments, the method can include providing at least a first prerecorded message related to toilet training; receiving a contact from a consumer; and enunciating the prerecorded message to the consumer. The prerecorded messages can include messages directed to the caregiver, the child, or both. The method can include providing a toilet training garment that includes a character graphic; and providing at least a first prerecorded message related to toilet training, the prerecorded message enunciated by a voice or image associated with the character graphic. The method can include providing a package containing a plurality of toilet training garments, wherein access information is included on the package, within the package, or on each toilet training garment.

### BACKGROUND OF THE INVENTION

The present invention relates to a method of assisting caregivers and children during the toilet training process, and more particularly to a method of enunciating a prerecorded message related to toilet training in response to a contact.

The toilet training process has long been a challenge for both parents and children. The toilet training process may incorporate a wide variety of different aspects, including many training techniques and training aids that may be used by parents and caregivers, hereinafter simply referred to as caregivers. One aspect of the total toilet training process is the change from diapers to training pants to help the child understand that he or she should now use the toilet just like adults. Another aspect of the total toilet training process can include accidents or other set-backs, which can be discouraging for both the caregiver and the child. Yet another aspect of the total toilet training process is successful completion of the process. In these and other aspects, positive encouragement, praise, and support are helpful to the training child. Such encouragement, praise, and support have traditionally been provided by the caregiver or other family member.

Further, many caregivers have difficulty in determining the readiness of a child to begin the toilet training process, and underestimate the difficulty of teaching the toilet training process to young children. If a child does not respond to an initial toilet training instruction or introduction, the caregiver can be at a loss for finding techniques, methods, or teaching tools to encourage the child to master the art of toilet training. Thus, while various teaching tools such as books, videotapes, charts with stickers, personalized toilets, and interactive toilet training kits are available, and while training pants and positive encouragement and support from the caregiver have been helpful in the toilet training process, there remains a need for new and improved educational and motivational mechanisms to facilitate the toilet training process.

### SUMMARY OF THE INVENTION

In response to the unmet needs set forth above, a new method of enunciating a prerecorded message related to toilet training in response to a contact has been invented.

In one embodiment, the method comprises providing at least a first prerecorded message related to toilet training; receiving a contact from a consumer, wherein the contact is received telephonically; and enunciating the prerecorded message to the consumer, wherein the prerecorded message is enunciated telephonically.

In another embodiment, the method comprises providing at least a first prerecorded message related to toilet training; receiving a contact from a consumer, wherein the contact is received via the internet; and enunciating the prerecorded message to the consumer, wherein the prerecorded message is enunciated via the internet, and wherein the message includes both audible and visible content.

In still another embodiment, the method comprises providing a toilet training garment that includes a character graphic; providing at least a first prerecorded message related to toilet training, the prerecorded message enunciated by a voice or image associated with the character graphic; receiving a contact from a consumer; and enunciating the prerecorded message to the consumer.

In yet another embodiment, the method comprises providing at least a first prerecorded caregiver message directed to assisting a caregiver with a toilet training process, and at least a first prerecorded child message directed to motivating a child during the toilet training process; receiving a contact from a consumer; prompting the consumer to make a message-type selection from among at least the first prerecorded caregiver message and the first prerecorded child message; receiving the message-type selection from the consumer; and enunciating the prerecorded message corresponding to the message-type selection.

In still another embodiment, the method comprises providing a package containing a plurality of toilet training garments, wherein access information is included at least one of on the package, within the package, or on each toilet training garment; providing at least three prerecorded child messages directed to motivating a child during a toilet training process, each prerecorded child message relating to a different stage of the toilet training process; providing at least three prerecorded caregiver messages directed to assisting a caregiver with the toilet training process, each prerecorded caregiver message relating to a different stage of the toilet training process; receiving a contact from a consumer; prompting the consumer to make a first selection from among at least the prerecorded child messages and prerecorded caregiver messages; subsequently prompting the consumer to make a second selection from among the at least three prerecorded messages corresponding to the first selection; and enunciating the prerecorded message corresponding to the second selection.

### BRIEF DESCRIPTION OF THE FIGURES

FIG. 1 is a flowchart depicting an exemplary embodiment of the method of the present invention.

FIG. 2 is a perspective front view of a toilet training garment suitable for use in conjunction with particular embodiments of the method of the present invention.

FIG. 3 is a perspective front view of a toilet training garment suitable for use in conjunction with particular embodiments of the method of the present invention.

### DETAILED DESCRIPTION OF PARTICULAR EMBODIMENTS

In particular embodiments, the method of the present invention includes providing one or more prerecorded messages related to toilet training. “Prerecorded” means previously recorded into or onto a message storage medium. Various media on which to record audio and/or video messages are well known in the art, and include such well known devices as magnetic tape, digital storage mechanisms, automated voice message systems, and the like. “Toilet training” refers to any activity or situation connected with learning to consistently urinate or defecate in a toilet or potty chair, and includes efforts to overcome enuresis. “Related to toilet training” means having some relation to the toilet training process, such as, for example, having instructional, educational, motivational, or encouraging aspects with respect to the subject of toilet training. The one or more messages can contain audible content, visual content, or both. In one example, one or more of the prerecorded messages is a song.

In particular embodiments, at least one of the prerecorded messages is a prerecorded child message. “Prerecorded child message” means a prerecorded message primarily intended to be heard or viewed by a child involved in toilet training, such as a message intended to encourage, motivate, or praise the child with respect to his or her participation in the toilet training process. Further, in particular embodiments, at least one of the prerecorded messages is a prerecorded caregiver message. “Prerecorded caregiver message” means a prerecorded message primarily intended to be heard or viewed by a caregiver, such as a message intended to educate the caregiver about the toilet training process, to provide toilet training tips or advice, or to offer/promote products or services related to toilet training.

In particular embodiments, one or more of the prerecorded messages can but need not be associated with a particular gender. “Associated with a particular gender,” “associated with a first gender,” “associated with a second gender,” and the like as used herein means that a message is tailored to or particularly suitable for a child of a particular gender. For example, in certain embodiments, a prerecorded child message is associated with a particular gender in that the message would be expected to be particularly well received by a child of that particular gender who hears or views the message. For instance, a message associated with boys can be articulated by a male voice or figure, and a message associated with girls can be articulated by a female voice or figure. This can be particularly advantageous in the toilet training process, as some research indicates that children are more receptive to toilet training guidance provided by an adult of the same gender. In a particular embodiment, the message is articulated by a popular children's character with which children of a particular gender relate especially well. For instance, boys are often excited about and interested in toy characters, cartoon characters, and movie characters that relate to subjects such as trains, racecars, space travel, power equipment, certain sports, or the like.

Girls, in contrast, are frequently excited about subjects such as dancing, dolls, dress-up activities, tea parties, certain fairytales, or the like. A prerecorded caregiver message can also be associated with a particular gender in that the prerecorded caregiver message would be adapted to deliver toilet training guidance or tips especially suitable for children of a particular gender.

In particular embodiments, one or more of the prerecorded messages relates to a particular stage in a toilet training process. For example, if a toilet training process is conceptually divided into the three general stages of (1) child about to begin or just beginning to toilet train, (2) child approximately midway through toilet training, and (3) child about to complete or having just completed toilet training, the method of the present invention can include at least three prerecorded messages, each one related to one of those three stages. In another example in which a toilet training process is conceptually divided into the three general stages of (1) determining that the child is ready to being toilet training based on observed signs of readiness, (2) making the switch from diapers to training pants, and (3) successfully coaching the child through the toilet training process, the method of the present invention can include at least three prerecorded messages, each one related to one of those three stages. In either example, the at least three messages can be either prerecorded caregiver messages, or prerecorded child messages. Alternatively, both prerecorded caregiver messages and prerecorded child messages can be provided that relate to the three stages in either example. Additional stages of a toilet training process can relate to, for example, a regressive stage or stage defined by setbacks, wherein the child is having difficultly maintaining previous success, or begins to or continues to wet his or her pants at day or night.

The method of the present invention further includes receiving a contact from a consumer. In one example, the contact is received telephonically. In another example, the contact is received via the internet. The method of the invention also includes enunciating at least one prerecorded message to a listener or viewer, such as a caregiver, a child, or both. The prerecorded message can be enunciated telephonically, via the internet, or by other suitable modes of communication. Note that as used herein, “enunciate” and conjugations thereof can refer to the articulation and/or display of audible and/or visual content. In certain embodiments, the one or more prerecorded messages can be stored on a medium that could be distributed to and retained by consumers (such as in response to a contact), such as a compact disc (CD) or digital video disc (DVD). The prerecorded messages could then be enunciated via a playback device owned by the consumer using the CD, DVD, or similar medium.

In particular embodiments, the method of the present invention includes prompting the consumer to make a gender selection signaling a selected gender. For example, prompting the consumer to make a gender selection can include a prompt that explicitly asks the consumer to select either male or female, either boy or girl, or the like. Alternatively, prompting the consumer to make a gender selection to signal a selected gender can be less direct. In one such example, the consumer can be prompted to choose between (1) hearing messages from a popular character with which boys typically relate, or (2) hearing messages from a popular character with which girls typically relate. In this way, a consumer's selection signals a selected gender without the consumer explicitly and directly selecting a particular gender.

In particular embodiments, the method of the present invention also includes receiving a gender selection from the consumer. In one example, the gender selection is received telephonically. For example, in certain embodiments, a consumer presses a button on a touchtone telephone to make a gender selection in response to a prompt. In other embodiments, a consumer vocalizes a selection into a telephone in response to a prompt. In another example, the gender selection is received via the internet. For example, in certain embodiments, a consumer presses a key, button, or combination of keys or buttons on a computer keyboard and/or mouse to make a gender selection in response to a prompt. In such embodiments, the prompt can include audible and/or visual content.

In embodiments in which a consumer makes a gender selection, the enunciating of the prerecorded message can include enunciating a message associated with the selected gender. For example, if the consumer makes a gender selection that signals the male gender, one or more messages associated with the male gender can be enunciated. Alternatively, if the consumer makes a gender selection that signals the female gender, one or more messages associated with the female gender can be enunciated.

In particular embodiments, the method of present invention includes prompting the consumer to input their child's name. For example, in certain embodiments, a consumer keys in the letters to her child's first name using a touchtone telephone. In another example, a consumer vocalizes her child's name into a telephone in response to a prompt. In another example, the child's name is input by a consumer via keys on a computer keyboard. By obtaining the child's name as part of particular embodiments of the method of present invention, subsequent prerecorded messages can be personalized via inclusion of the child's name, such as by the enunciation of a prerecorded articulation of the child's name.

In particular embodiments, the method of the present invention includes prompting the consumer to make a stage selection signaling a selected stage of the toilet training process. For example, prompting the consumer to make a stage selection can include a prompt that explicitly asks the consumer to select from among specific toilet training stages, such as those discussed above. Alternatively, prompting the consumer to make a stage selection to signal a selected stage of the toilet training process can be less direct, such as by asking the consumer a series of questions concerning their child's behavior or their child's level of involvement or progress in toilet training. One suitable example of a method to determine a child's level of progress in the toilet training process is outlined in U.S. Pat. No. 6,250,929 issued Jun. 26, 2001 to Kolb et al. and assigned to Kimberly-Clark Worldwide, Inc., the entirety of which is incorporated by reference herein to the extent it is consistent herewith.

In particular embodiments, the method of the present invention also includes receiving a stage selection from the consumer. In one example, the stage selection is received telephonically. For example, in certain embodiments, a consumer presses a button on a touchtone telephone to make a stage selection in response to a prompt. In other embodiments, a consumer vocalizes a selection into a telephone in response to a prompt. In another example, the stage selection is received via the internet. For example, in certain embodiments, a consumer presses a key, button, or combination of keys or buttons on a computer keyboard and/or mouse to make a stage selection in response to a prompt. In such embodiments, the prompt can include audible and/or visual content.

In embodiments in which a consumer makes a stage selection, the enunciating of the prerecorded message can include enunciating a message that relates to the selected stage. For example, if the consumer makes a stage selection that signals that the child is just beginning the toilet training process, one or more messages that relate to beginning the toilet training process can be enunciated.

In particular embodiments, the method of the present invention includes prompting the consumer to make a message-type selection from among two or more message types. For example, a consumer can be prompted to choose to hear either prerecorded caregiver messages or prerecorded child messages. Certain embodiments of the invention can further include receiving a message-type selection from the consumer. In one example, the message-type selection is received telephonically. In another example, the message-type selection is received via the internet as described above in relation to the receipt of other selections. In embodiments in which a consumer makes a message-type selection, the enunciating of the prerecorded message can include enunciating a message that relates to the selected message-type. For example, if the consumer selects caregiver-type messages, one or more caregiver messages can be enunciated. Likewise, if the consumer selects child-type messages, one or more child messages can be enunciated.

In particular embodiments, method of the present invention includes prompting the consumer to choose among two or more language selections. For example, the first message can prompt the consumer to choose a language, such as English, Spanish, or French, in which the succeeding messages will be articulated.

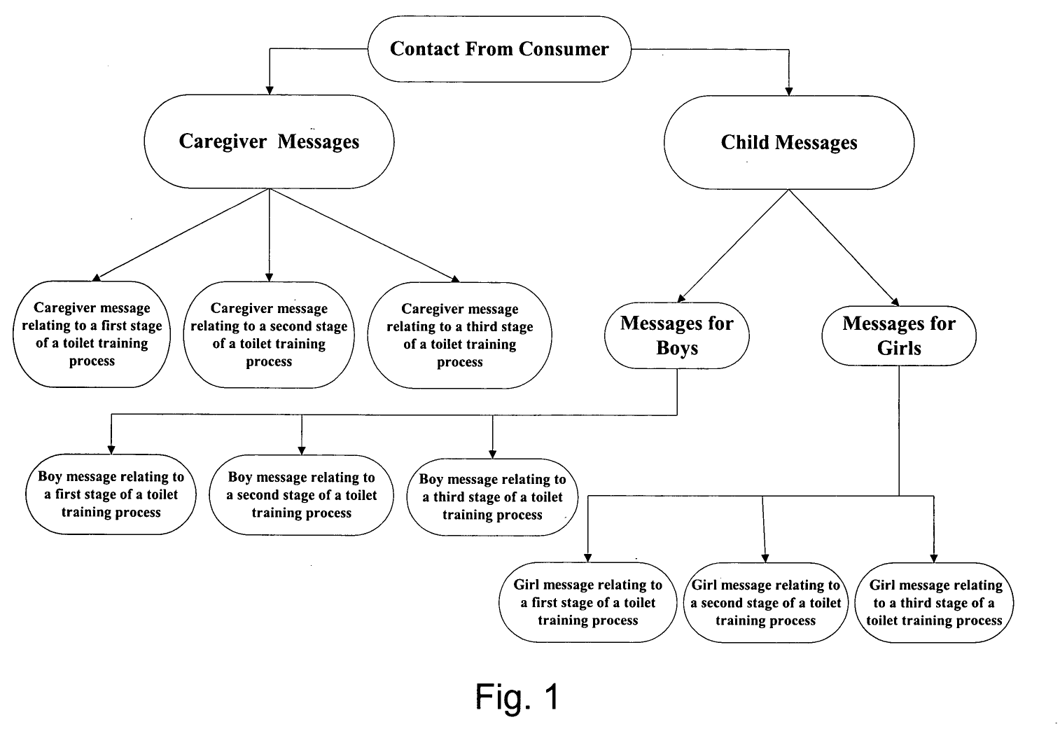

It should be noted that various combinations and subcombinations of the above features of the present method are possible. For example, one embodiment of the method of the present invention, representatively illustrated in the form of a flowchart in FIG. 1, includes providing both prerecorded caregiver messages and prerecorded child messages, and prompting the consumer to choose between those two types following receipt of the initial contact by the consumer. The embodiment can further include two or more caregiver messages, such as three caregiver messages, each of which relates to a different stage of a toilet training process. The embodiment can further include two or more child messages, each of which relates to a different gender. The embodiment can further include two or more child messages, such as three child messages, associated with the male gender, each such message relating to a different stage of a toilet training process. The embodiment can further include two or more child messages, such as three child messages, associated with the female gender, each such message relating to a different stage of a toilet training process. In other embodiments, the child messages relate equally to either gender. In particular embodiments, the child messages are in the form of a song.

In particular embodiments, the method of the present invention further includes providing a toilet training garment that includes a character graphic. The term “character graphic” is used herein to refer to a graphic containing an anthropomorphous image, and in particular an image having or suggesting human form or appearance which ascribes human motivations, characteristics or behavior to inanimate objects, animals, natural phenomena, cartoon characters, or the like. With respect to children's training pants, a character graphic would ideally be suitable for children's underwear and could be utilized to motivate children to wear the training pants and use a potty or toilet. To that end, a character graphic can be associated with popular characters in the media, advertising or well known in a particular culture. The role of the character graphic can be to help a child feel like a BIG KID® (a registered trademark of Kimberly-Clark Corporation) and to motivate them to become toilet trained. The character graphic can also give parents and caregivers an interactive element for use during toilet training. Further, the character graphic may provide a source of comfort for the child and a buddy who reduces stress during the training period. The character graphic can, in particular embodiments, comprise a portion of a multicolored scene graphic that sets up a theme for the illustrated scene. As such, the character graphic can provide an opportunity for educational interaction between the child and the parent or caregiver. More specifically, the parent or caregiver can use the graphic story-line to make up a game or story for the purpose of toilet training progress.

Suitable character graphics can include animals, people, inanimate objects, natural phenomena, cartoon characters, or the like that can or can not be provided with human features such as arms, legs, facial features or the like. For purposes of enhanced toilet training, it may be desirable for the character graphic to be familiar to the child, such as an identifiable cartoon character. The character graphics should at least be a type that the child can relate to, examples of which could include animals, toys, licensed characters, or the like. Character graphics can be made more personable and friendly to the child by including human-like features, human-like expressions, apparel, abilities, or the like. By way of illustration, an animal character graphic can be shown smiling, wearing clothing, or involved in or performing some activity, such as playing sports, fishing, driving, playing with toys, having a tea party, or the like. In particular embodiments, the character graphic can desirably be created to project an appearance that could be described as friendly, positive, non-intimidating, silly, independent, inspirational, active, expressive, dauntless and/or persevering.

The training garment desirably comprises one or more appearance-related components. Examples of appearance-related components include, but are not limited to, graphics; highlighting or emphasizing leg and waist openings in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering active graphics, temperature indicators, and the like in the product; registering a back label, or a front label, in the product; and registering written instructions at a desired location in the product.

In particular embodiments, the toilet training garment that is provided in certain embodiments of the method of the present invention includes at least one active graphic. The term “active graphic” as used herein refers to an appearing graphic, a fading graphic, or a combination of appearing and fading graphics. The term “appearing graphic” is used herein to refer to a graphic that becomes visible or becomes significantly more visible when exposed to urine, or that becomes visible or becomes significantly more visible with the passage of time when exposed to the environment but not exposed to urine. Conversely, the term “fading graphic” is used herein to refer to a graphic that becomes invisible or significantly less visible when exposed to urine, or that becomes invisible or significantly less visible with the passage of time when exposed to the environment but not exposed to urine. Examples of toilet training garments suitable for use with particular embodiments of the present invention, including toilet training garments that have character graphics and active graphics, are disclosed in U.S. Pat. No. 6,297,424 issued Oct. 2, 2001 to Olson et al. and U.S. Pat. No. 6,307,119 issued Oct. 23, 2001 to Cammarota et al, both assigned to Kimberly-Clark Worldwide, Inc., and both of which are incorporated by reference herein to the extent they are consistent herewith.

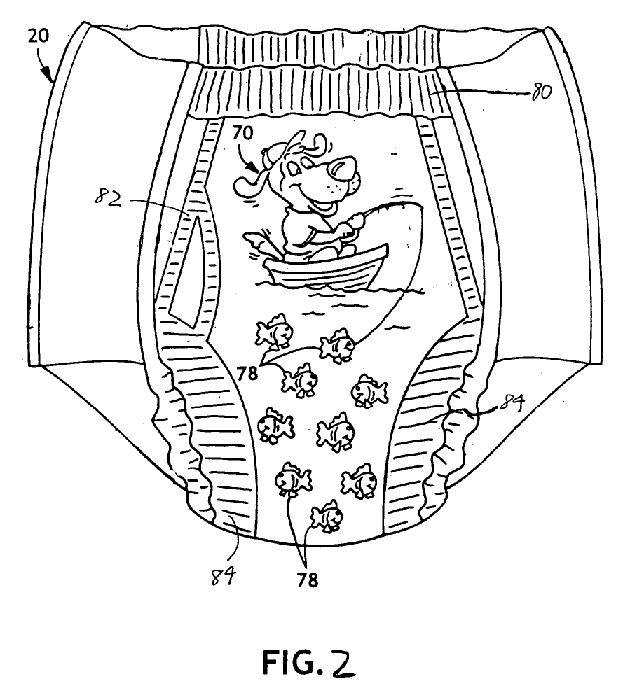

One embodiment of a training pant designed for use by young boys is representatively illustrated in FIG. 2 and designated generally by reference numeral 20. The training pant 20 includes graphics, including active, wetness-indicating graphics 78. More specifically, the training pant 20 includes a character graphic 70 in the form of a dog having human-like expressions and wearing a shirt and a hat, graphics depicting a boat, water, and a fishing pole, and a plurality of active graphics 78 representing fish. The graphics also include a simulated elastic waistband 80, a simulated fly opening 82, and simulated elastic leg bands 84.

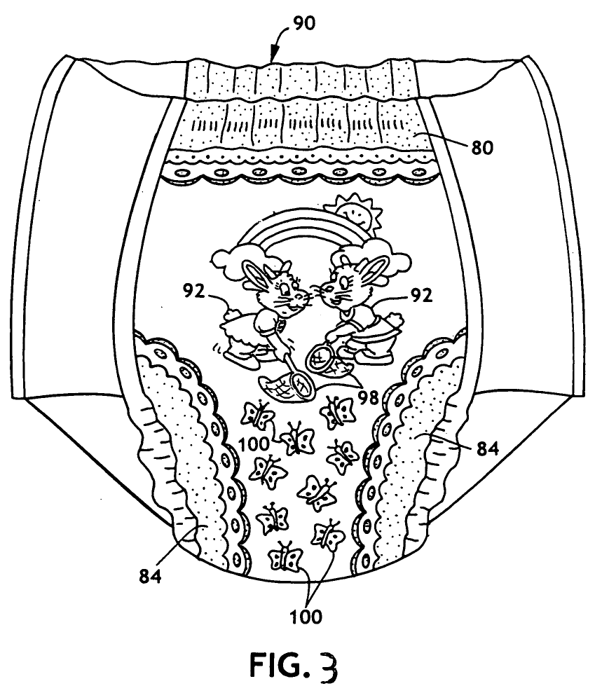

An embodiment of a training pant designed for use by young girls is representatively illustrated in FIG. 3 and designated generally by reference numeral 90. The illustrated training pant 90 includes character graphics 92, graphics in the form of clouds, rainbow and sun, and butterfly nets, respectively, and a plurality of active graphics 100 representing butterflies. The also include a simulated elastic waistband 80 with ruffles, and simulated elastic leg bands 84 with ruffles.

In particular embodiments, the method of the present invention includes providing a prerecorded message articulated by a voice associated with the character graphic depicted on the toilet training garment provided to the consumer. “Enunciated by a voice or image associated with a character graphic” and the like means that the message is articulated by a voice that children would perceive to be the voice of the depicted character, or that the message is presented by an image, such as an animated image, that children would perceive to be the depicted character. In certain embodiments, the character voice or character image is associated with a particular gender. For example, in an embodiment in which the consumer has made a gender selection, a message can be articulated by a voice which is that of a speaker of the selected gender. For example, if the character graphic is a well known male sports figure, the consumer can make a gender selection that signals the male gender, and a prerecorded child message can be articulated by a voice that is or that resembles the voice of the well known male sports figure. In certain embodiments, the character graphic is an animal, and one or more prerecorded messages are articulated by a voice resembling the sound of the animal.

In embodiments of the present invention in which a toilet training garment includes active graphics, the prerecorded message can be adapted to encourage a child to maintain the active graphic 40 in an inactivated condition. For example, in an embodiment in which the toilet training garment 20 includes active graphics 78 in the form of disappearing fish as representatively illustrated in FIG. 2, a prerecorded child message such as, “Keep up the good work with your potty training, buddy, and don't forget to keep the fish showing!” can be articulated. Further, the message could be articulated by a voice resembling the sound a dog might make. In this way, the voice of the prerecorded message is associated with the dog character graphic 70.

The toilet training garment can be provided to the consumer in a variety of ways. Desirably, the garment is suitably packaged for sale or other distribution. Such packaged garments can be sold in stores, via the internet, or by other methods of sale or distribution as are well known in the art. In particular embodiments, the toilet training garments that are provided in certain embodiments of the present method are provided in a package. In such embodiments, access information can be included on the package, within the package, on one or more toilet training garments within the package, or combinations thereof. “Access information” means information that instructs a consumer how to access the one or more prerecorded messages provided in accordance with the method of the present invention. Such access information can be provided on the package, such as printed on a plastic packaging bag or on a leaflet or coupon attached to the bag; within the package, such as printed on a leaflet or coupon placed inside the bag; or printed on one or more individual garments residing within the package. Examples of access information include, but are not limited to, a telephone number and an internet website address. Access information can be provided other than on the package, such as via a website, email, direct mail, newspaper advertising, television or radio commercials, or other media.

### EXAMPLE

The following example constitutes a “script” containing various prompts and prerecorded messages that form one possible embodiment of the method of the present invention. This particular embodiment contemplates primarily telephonic communication. The example makes reference to a consumer (e.g., mother) experiencing the toilet training process with her son or daughter, though the example is of course equally suitable to other caregivers, such as a day care provider. In this example, a package of either boy or girl toilet training garments, such as a bag of HUGGIES PULL-UPS training pants, available from Kimberly-Clark Corporation, Neenah, Wis., U.S.A., is sold to a consumer in a retail store. Each training pant garment includes a character graphic thereon, and it is contemplated in this particular example that the characters on the boy training pants are different from the characters on the girl training pants. A toll-free access telephone number is printed on each bag. When the consumer calls the access number, she hears the following prerecorded message:
Thank you for calling the PULL-UPS Potty Training TOTLINE. At PULL-UPS, we know every child trains differently, so we designed the TOTLINE as a tool to help you and your child as you go through the potty training process together.
   The consumer then hears the following prerecorded message to prompt the consumer to make a message-type selection:
The following prompts will direct you to encouraging messages for your child or potty training tips for adults involved in the process.
Press 1 for messages for kids from the character on your child's training pant.
Press 2 for tips for parents on potty training.
   If the consumer makes a message-type selection corresponding to prerecorded child messages (in other words, if the consumer presses 1), the consumer subsequently hears the following prerecorded message to prompt the consumer to make a gender selection:
Press 1 for messages for boys from [popular children's character present on the boy training pants].
Press 2 for messages for girls from [popular children's character present on the girl training pants].
   Following receipt of a gender selection from the consumer, the consumer hears a series of prerecorded messages to prompt the consumer to make a stage selection from among three stages of a toilet training process. For example, if the consumer makes a male gender selection, the consumer will hear the following prerecorded messages:
Press 1 for a message from [popular children's character present on the boy training pants] if your child is just beginning potty training.
Press 2 for a message from [popular children's character present on the boy training pants] if your child needs encouragement or motivation for potty training.
Press 3 for a message from [popular children's character present on the boy training pants] for your child if he has completed potty training.
   If the consumer makes a stage selection signaling that her son is about to begin or just beginning to toilet train (in other words, if the consumer presses 1), the consumer's son will hear the following message:
Greetings, friend! This is [popular children's character present on the boy training pants]. It looks like you're ready to take the next step in becoming a big boy—potty training! Good luck and keep up the good work. I'm so happy you are ready to begin potty training!
   Alternatively, if the consumer makes a stage selection signaling that her son is approximately midway through toilet training (in other words, if the consumer presses 2), the consumer's son will hear the following message:
Greetings, friend! It's me, your friend [popular children's character present on the boy training pants]. Take it from a guy like me—potty training can be tough, but you can do it. I know you can. Keep up the good work!
   Alternatively, if the consumer makes a stage selection signaling that her son has just completed toilet training, (in other words, if the consumer presses 3), the consumer's son will hear the following message:
Greetings, friend! This is [popular children's character present on the boy training pants], saying, “Good for you, pal—you're potty trained! You sure are a big boy!
   If, instead, the consumer makes a female gender selection, the consumer will hear the following prerecorded messages:
Press 1 for a message from [popular children's character present on the girl training pants] if your child is just beginning potty training.
Press 2 for a message from [popular children's character present on the girl training pants] if your child needs encouragement or motivation for potty training.
Press 3 for a message from [popular children's character present on the girl training pants] for your child if she has completed potty training.
   If the consumer makes a stage selection signaling that her daughter is about to begin or just beginning to toilet train (in other words, if the consumer presses 1), the consumer's daughter will hear the following message:
Oh, hello, my name is [popular children's character present on the girl training pants]. I'm very happy you are ready to start potty training! You're becoming a big girl so fast! Good luck—I know you can do it.
   Alternatively, if the consumer makes a stage selection signaling that her daughter is approximately midway through toilet training, (in other words, if the consumer presses 2), the consumer's daughter will hear the following message:
Oh, hello, it's me, your friend [popular children's character present on the girl training pants]. A little bird told me that you're a potty training princess. Keep trying—I know you can do it.
   Alternatively, if the consumer makes a stage selection signaling that her daughter has just completed toilet training, (in other words, if the consumer presses 3), the consumer's daughter will hear the following message:
Hello, it's me, [popular children's character present on the girl training pants]. Congratulations! I knew you'd be potty trained in no time at all. Why, you're a potty trained princess and a big girl too!
   If, at the message-type selection point of the method of the present example, the consumer makes a message-type selection corresponding to prerecorded caregiver messages (in other words, if the consumer presses 2), the consumer subsequently hears the following prerecorded message to prompt the consumer to make a stage selection:
The following prompts and messages will help you, as the child's caregiver, get successfully through the potty training process.
Are we ready yet? Press 1 to learn about signs that your child is ready to begin potty training.
Do you have to go? Press 2 if your child is ready to make the switch out of diapers into disposable training pants.
Are we there yet? Press 3 to learn about being your child's partner through praise and support.
   If the consumer makes a stage selection signaling that she would like help in determining that her child is ready to be toilet trained based on observed signs of readiness (in other words, if the consumer presses 1), the consumer will hear the following message:
Hi. Look for two or more of the following signs that indicate it's time to start potty training:
1) Staying dry for at least two hours or during a nap.
2) Wanting wet or soiled diapers changed.
3) Understanding and cooperating with simple instructions.
4) Showing interest in the toilet.
5) Indicating the need to go potty with words, posture or facial expression.
6) Wanting to wear training pants or underwear.
   If the consumer makes a stage selection signaling that she would like help in determining that her child is ready to make the switch from diapers to training pants (in other words, if the consumer presses 2), the consumer will hear the following message:
Hi. Congratulations! Here are some tips to help make the switch from diapers to training pants:
1) Make a big deal of the fact that training pants are just like real underwear.
2) Teach your child to pull them up and down all by himself or herself.
3) Explain the difference between wet and dry and encourage your child to keep the designs on his or her PULL-UPS training pants dry so they don't disappear.
4) Go shopping with your child and let him pick out his first pair of real underwear. Explain to him that wearing PULL-UPS training pants will help get him ready to wear his real underwear.
5) Change your child's PULL-UPS training pants immediately after he wets them to teach him that being dry is best.
   If the consumer makes a stage selection signaling that she would like to learn about successfully coaching her child through the toilet training process through praise and support (in other words, if the consumer presses 3), the consumer will hear the following message:
Hi. You're almost there! Celebrate your child's accomplishments to reinforce progress. Stumped for rewards? Try these:
1) Putting a stamp or sticker on a progress chart.
2) Playing a favorite tape or CD.
3) Reading a favorite story.
4) Giving a big hug.
   At the end of every prerecorded message outlined above, the following prerecorded message can be enunciated:
Press 9 to return to the introduction menu, or call [toll-free access number].

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. For example, features described in relation to one embodiment may be incorporated into any other embodiment of the invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments such as the Example outlined above, yet the absence of a particular advantage shall not be construed to necessarily mean that such an embodiment is outside the scope of the present invention.

1. A method of enunciating a prerecorded message related to toilet training in response to a contact, comprising:
providing at least a first prerecorded message related to toilet training;
receiving the contact from a consumer, wherein the contact is received telephonically; and
enunciating the prerecorded message to the consumer, wherein the prerecorded message is enunciated telephonically.

2. A method of enunciating a prerecorded message related to toilet training in response to a contact, comprising:
providing at least a first prerecorded message related to toilet training;
receiving the contact from a consumer, wherein the contact is received via the internet; and
enunciating the prerecorded message to the consumer, wherein the prerecorded message is enunciated via the internet, and wherein the message includes both audible and visible content.

3. A method of enunciating a prerecorded message related to toilet training in response to a contact, comprising:
providing a toilet training garment that includes a character graphic;
providing at least a first prerecorded message related to toilet training, the prerecorded message enunciated by a voice or image associated with the character graphic;
receiving the contact from a consumer; and
enunciating the prerecorded message to the consumer.

4. The method of claim 3, further comprising:
providing at least a second prerecorded message, wherein the first prerecorded message is associated with a first gender, and the second prerecorded message is associated with a second gender;
prompting the consumer to make a gender selection signaling a selected gender from among the first and second genders; and
receiving the gender selection from the consumer,
wherein the enunciating comprises enunciating the message associated with the selected gender.

5. The method of claim 3, further comprising:
providing at least a second prerecorded message, wherein the first prerecorded message relates to a first stage of a toilet training process, and the second prerecorded message relates to a second stage of the toilet training process;
prompting the consumer to make a stage selection signaling a selected stage of the toilet training process from among at least the first and second stages; and
receiving the stage selection from the consumer,
wherein the enunciating comprises enunciating the message relating to the selected stage.

6. The method of claim 5, further comprising:
providing at least third and fourth prerecorded messages, wherein the third prerecorded message relates to the first stage of the toilet training process, and the fourth prerecorded message relates to the second stage of the toilet training process, and further wherein the first and second messages are associated with a first gender, and the third and fourth messages are associated with a second gender;
prompting the consumer to make a gender selection signaling a selected gender from among the first and second genders; and
receiving the gender selection from the consumer,
wherein the enunciating further comprises enunciating the message associated with the selected gender.

7. The method of claim 6, wherein the prerecorded message is articulated by a voice, and the voice is that of a speaker of the selected gender.

8. The method of claim 3, wherein the contact is received telephonically, and the prerecorded message is enunciated telephonically.

9. The method of claim 3, wherein the contact is received via the internet, and the prerecorded message is enunciated via the internet, wherein the message includes both audible and visible content.

10. The method of claim 3, wherein the toilet training garment includes at least one active graphic, and the prerecorded message is adapted to encourage a child to maintain the active graphic in an inactivated condition.

11. The method of 3, wherein the toilet training garment is provided in a package, and access information is included at least one of on the package, within the package, or on the toilet training garment.

12. A method of enunciating a prerecorded message related to toilet training in response to a contact, comprising:
providing at least a first prerecorded caregiver message directed to assisting a caregiver with a toilet training process, and at least a first prerecorded child message directed to motivating a child during the toilet training process;
receiving the contact from a consumer;
prompting the consumer to make a message-type selection from among at least the first prerecorded caregiver message and the first prerecorded child message;
receiving the message-type selection from the consumer; and
enunciating the prerecorded message corresponding to the message-type selection.

13. The method of claim 12, further comprising:
providing at least a second prerecorded caregiver message directed to assisting a caregiver with the toilet training process, wherein the first prerecorded caregiver message relates to a first stage of the toilet training process, and the second prerecorded message relates to a second stage of the toilet training process;
prompting the consumer to make a stage selection signaling a selected stage of the toilet training process from among at least the first and second stages; and
receiving the stage selection from the consumer,
wherein the enunciating comprises enunciating a prerecorded caregiver message relating to the selected stage.

14. The method of claim 12, further including providing a toilet training garment that includes a character graphic, wherein the toilet training garment is provided in a package, and access information is included at least one of on the package, within the package, or on the toilet training garment.

15. The method of claim 12, wherein the prerecorded message is enunciated via the internet, and wherein the message includes both audible and visible content.

16. A method of enunciating a prerecorded message related to toilet training in response to a contact, comprising:
providing a package containing a plurality of toilet training garments, wherein access information is included at least one of on the package, within the package, or on each toilet training garment;
providing at least three prerecorded child messages directed to motivating a child during a toilet training process, each prerecorded child message relating to a different stage of the toilet training process;
providing at least three prerecorded caregiver messages directed to assisting a caregiver with the toilet training process, each prerecorded caregiver message relating to a different stage of the toilet training process;
receiving the contact from a consumer;
prompting the consumer to make a first selection from among at least the prerecorded child messages and prerecorded caregiver messages;
subsequently prompting the consumer to make a second selection from among the at least three prerecorded messages corresponding to the first selection; and
enunciating the prerecorded message corresponding to the second selection.

17. A method of enunciating a prerecorded message related to toilet training in response to a contact, comprising:
providing a package containing a plurality of toilet training garments, each training garment including a character graphic, and wherein access information is included at least one of on the package, within the package, or on each toilet training garment;
providing at least three prerecorded child messages directed to motivating a child during a toilet training process, each prerecorded child message relating to a different stage of the toilet training process, and each prerecorded child message enunciated by a voice or image associated with the character graphic;
providing at least three prerecorded caregiver messages directed to assisting a caregiver with the toilet training process, each prerecorded caregiver message relating to a different stage of the toilet training process;
receiving the contact from a consumer;
prompting the consumer to make a first selection from among at least the prerecorded child messages and prerecorded caregiver messages;
subsequently prompting the consumer to make a second selection from among the at least three prerecorded messages corresponding to the first selection; and
enunciating the prerecorded message corresponding to the second selection.

18. The method of claim 17, wherein the contact is received telephonically, and each prerecorded child message is articulated by a voice associated with the character graphic.
